(19) United States
(12) Patent Application Publication
(10) Pub. No.: US 2026/0253509 A1
(43) Pub. Date: Aug. 27, 2026

(54) SYSTEMS AND METHODS FOR CLINICAL PROCEDURE TRAINING USING MIXED ENVIRONMENT TECHNOLOGY

(71) Applicant: University of Cincinnati, Cincinnati, OH (US)

(72) Inventors: Lisa M. Hachey, Cincinnati, OH (US); Tamara Pavlik-Maus, Cincinnati, OH (US)

(73) Assignee: University of Cincinnati, Cincinnati, OH (US)

(21) Appl. No.: 18/875,810

(22) PCT Filed: Jun. 28, 2023
(86) PCT No.: PCT/US2023/026441

§ 371 Date: Dec. 17, 2024

Related U.S. Application Data
(60) Provisional application No. 63/356,462, filed on Jun. 28, 2022.

(51) Int. Cl. G09B 19/00; G06F 3/01; G06V 10/80; G06V 10/82

(52) U.S. Cl. CPC G09B 19/00; G06F 3/011; G06F 3/016; G06V 10/80; G06V 10/82

(57) ABSTRACT

Embodiments are systems for clinical procedure training including a physical model of an anatomic region including a position sensor and a haptic sensor, a medical tool operable to interact with the physical model, a camera, a display, a processor, and a computer-readable medium. The computer-readable medium stores computer-readable instructions that cause the processor to acquire, using the camera, image data of the physical model and the medical tool, detect, using the position sensor, a position of the medical tool during an operation associated with the anatomic region by a user, detect, using the haptic sensor, an applied pressure exerted on the physical model, generate a real-time virtual representation by overlaying an anatomic image on the physical model or the medical tool, display the real-time virtual representation on the display, and provide feedback to the user based on the position and the applied pressure.

### CROSS REFERENCE TO RELATED APPLICATION

This application claims priority to U.S. Provisional Application Ser. No. 63/356,462, filed Jun. 28, 2022, the entire contents of which are incorporated herein by reference.

### TECHNICAL FIELD

The present disclosure relates to medical simulation, and more particularly, to medical simulation for clinical skills training.

### BACKGROUND

Academic institutions have recently developed online educational programs that have transformed and globalized distance education. With the help of technology, students and faculty can now interact asynchronously to meet student learning objectives and program-specific credentialing requirements. However, for health profession students, clinical skills are a vital aspect of their education to become proficient in specific skill performance. Learning based on experiences is essential. Unfortunately, current distance learning programs are limited to training videos, video streaming, and didactic content. This type of learning does not provide the physical motor skills training required for many health occupations. As a result, the need for remote clinical skills training systems remains unfulfilled.

### SUMMARY

In a first aspect, a system for clinical procedure training includes a physical model of an anatomic region including a position sensor and a haptic sensor, a medical tool operable to interact with the physical model, a camera, a display, a processor, and a computer-readable medium. The computer-readable medium stores computer-readable instructions that cause the processor to acquire, using the camera, image data of the physical model and the medical tool, detect, using the position sensor, a position of the medical tool during an operation associated with the anatomic region by a user, detect, using the haptic sensor, an applied pressure exerted on the physical model, generate a real-time virtual representation by overlaying an anatomic image on the physical model or the medical tool, display the real-time virtual representation on the display, and provide feedback to the user based on the position and the applied pressure.

In a second aspect, a method for clinical procedure training includes acquiring, using a camera, image data of a physical model of an anatomic region and a medical tool operable to interact with the physical model, detecting, using a position sensor, a position of the medical tool during an operation associated with the anatomic region by a user, detecting, using a haptic sensor, an applied pressure exerted on the physical model, generating a real-time virtual representation by overlaying an anatomic image on the physical model or the medical tool, displaying the real-time virtual representation on a display, and providing feedback, based on the position and the applied pressure, to the user.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an exemplary clinical procedure training system of the present disclosure, according to one or more embodiments shown and described herein;

FIG. 2 schematically depicts exemplary non-limiting components of the clinical procedure training system of the present disclosure, according to one or more embodiments shown and described herein;

FIG. 3A schematically depicts an exemplary physical view of the operation of a medical tool on a physical model of the present disclosure, according to one or more embodiments shown and described herein;

FIG. 3B schematically depicts an exemplary mixed reality view of the operation of a medical tool on a physical model of the present disclosure, according to one or more embodiments shown and described herein;

FIG. 4 schematically depicts an exemplary shared mixed reality view of the operation of a medical tool on a physical model of the present disclosure, according to one or more embodiments shown and described herein;

FIG. 5 schematically depicts an exemplary user interface of the clinical procedure training system of the present disclosure, according to one or more embodiments shown and described herein;

FIG. 6 schematically depicts an exemplary block diagram a the method for clinical procedure training, according to one or more embodiments shown and described herein;

FIG. 7 schematically depicts an exemplary block diagram of the method for continuously training the clinical procedure training system, according to one or more embodiments shown and described herein;

FIG. 8 illustrates a flow diagram of illustrative steps for the clinical procedure training of the present disclosure, according to one or more embodiments shown and described herein; and

FIG. 9 illustrates a flow diagram of illustrative steps for the multiparty clinical procedure training of the present disclosure, according to one or more embodiments shown and described herein.

### DETAILED DESCRIPTION

The present disclosure involves a clinical procedure training system for developing clinical skills training in distance learning programs by incorporating mobile-learning and virtual technologies. This will enable students to develop personal, technical, and clinical skills desirable for the transition into the advanced practice role. This invention applies Augmented Reality (AR), Mixed Reality (MR), Extended Reality (XR or X-reality), holography (image overlay), and artificial intelligence (AI) in immersive virtual worlds involving information exchange (sensory perception or otherwise) between multiple parties across distance/space in remote asynchronous or synchronous environments.

In one embodiment, the training system includes a structurally truthful simulation physical model of an anatomic region to appropriately perform a clinical skill or procedure. In one embodiment, the physical model may incorporate strategically placed sensors and haptic feedback elements or similar sensors, on a variety of physical models and instruments. These sensors may note placement and measure pressure so limits in a target structure are noted. Accuracy and feedback for comparative data on user performance may be collected as well as data for comparative analysis against a group.

In another embodiment, the training system includes an augmented reality (AR) or mixed reality (MR) computer simulation of the anatomic region that complements the physical model. The simulation associates an image of at least the anatomic region with the physical model. In some embodiments, the invention incorporates a device such as a cell phone or tablet that displays a two dimensional (2D) representation of the simulated camera view of the current instrument location and trajectory, providing real-time feedback on the device screen. The application tracks placement, position, and instrument to tissue pressure of instrumentation and/or devices within the physical model and provides relevant feedback as designated by the procedure objectives. An artist or graphic rendition of the anatomical structures may be displayed on the mobile device.

In another embodiment, the training system includes a display device that displays the computer simulation to a user. In some embodiments, digital augmented elements can be displayed superimposed on the real world through a display via head-mounted eyewear, such as Microsoft HoloLens (Microsoft Corp., Washington, US) or the like. In another embodiment, sensors and tracking software are used to line up overlaid hologram images and stabilize the image for the user's movement within the environment.

In one embodiment, the training system includes one or more instruments that are manipulated by the user to interact with the physical model. In another embodiment, the instruments comprise at least one sensor. Various sensors may be used, including, without limitations, position sensors, light sensors, haptic sensors, temperature sensors, accelerometers, gyroscopes, magnetic sensors, pH sensors, oxygen sensors, and sound sensors.

In another embodiment, the training system includes a learning model that incorporates games to train the user. The learning model can be one-to-one with the instructor or one-to-many learners at the same time (synchronous). The learning model can also be one instructor with multiple groups of one or more learners with each group learning the same skill at different times (asynchronous). The learning model can include co-location of instructor and learners (local) or distance separated (remote). Software such as artificial intelligence (“AI”) can be used to interact with the model/target structure during the skills and/or procedure performance. The AI may include skill specific vocabulary developed with the ability to “learn” as language expands. In some embodiments, game designs are used for evaluation in real-time and across time, comparative within groups and between groups, and assessing the educational information and delivery mechanisms of training. Data points (mining) may be used to assess the user, groups and training. In one embodiment, the computer simulation AI simulates physiological responses to the user's interaction with the physical model.

Various embodiments of the methods and systems for clinical procedure training are described in more detail herein. Whenever possible, the same reference numerals will be used throughout the drawings to refer to the same or like parts.

As used herein, the singular forms “a,” “an” and “the” include plural referents unless the context clearly dictates otherwise. Thus, for example, reference to “a” component includes aspects having two or more such components unless the context clearly indicates otherwise.

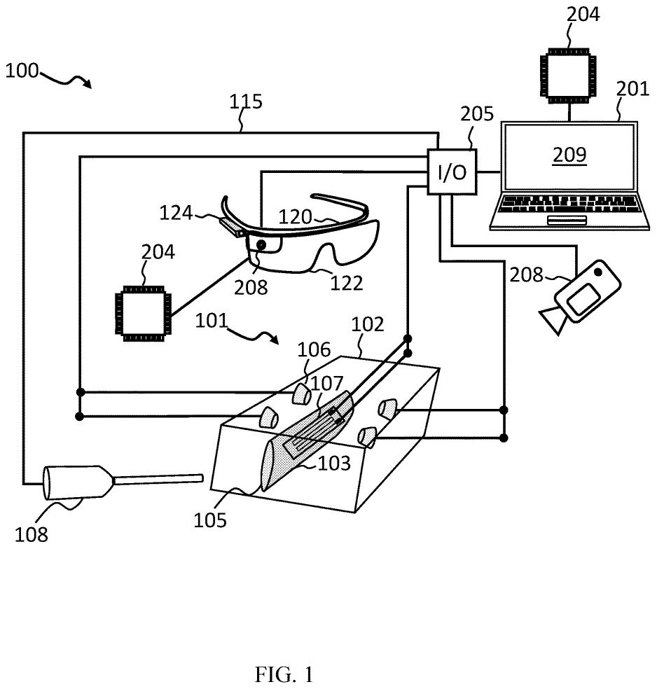

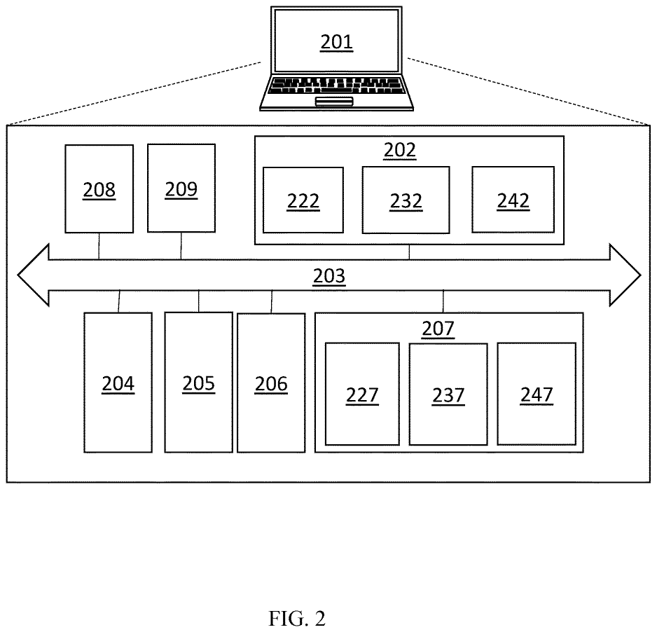

Turning to the figures, FIG. 1 schematically depicts an example clinical procedure training system of the present disclosure. The clinical procedure training system 100 includes a physical model 101 of an anatomic region, a medical tool 108 operable to interact with the physical model 101, a camera 208, a display 209, and a processor 204 (e.g. as illustrated in FIG. 2). The clinical procedure training system may further include a controller 201 having the display 209, input/output (I/O) hardware 205, and connections 115. The connections 115 connect components of the clinical procedure training system 100 and allow signal transmission between the components of the clinical procedure training system. For example, the connections 115 may connect the sensors in the physical model 101 and the medical tool 108, the camera 208 to the controller 201 at the I/O hardware 205. The connections 115 may be wired or wireless.

The disclosed embodiments may include a physical model 101. A physical model is a three-dimensional representation or replica of a specific area or structure of the human body. In embodiments, the physical model 101 may be, without limitation, a skeletal anatomy model, a muscular anatomy model, an organ anatomy model, a skull anatomy model, a torso anatomy model, a joint model, a vascular model, or a full-body anatomical model. In a non-limiting example, the physical model 101 in FIG. 1 may represent a pelvic model. More specifically, the physical model 101 in FIG. 1 may represent a female pelvic model.

As illustrated in FIG. 1, the pelvic model as a physical model 101 includes a canal area 103 inside the pelvic model. The canal area 103 has an opening 105 at a surface 102 of the pelvic model. The canal area 103 may include a simulated vaginal canal, a simulated cervical canal, a simulated uterine cavity, and simulated fallopian tubes. A cervical canal is the passageway that extends through the cervix, connecting the uterine cavity to the vaginal canal. The simulated cervical canal may serve as a canal for the insertion of a medical tool, such as an intrauterine device (IUD) tool or a tenaculum during gynecological procedures. The simulated vaginal canal may serve as a model for the insertion of a variety of medical tools, including a speculum, forceps, tenaculum, ultrasound probe, and the like during gynecological examinations or procedures. A uterine cavity is the interior space within the uterus. Fallopian tubes, also known as uterine tubes, are narrow, canal-like structures that extend from the upper uterus, or uterine horns, towards the ovaries. The opening 105 may simulate a vaginal orifice as the opening of the vagina, which serves as the entrance into the canal area 103.

The physical model 101 may include one or more position sensors 106. The position sensors 106 may detect the position of a medical tool 108 or other objects that is transmitted to or interacts with the physical model 101. The position sensors 106 may be, without limitation, an optical position sensor, a magnetic position sensor, an ultrasonic position sensor, a capacitor position sensor. An optical position sensor uses light-based technology to determine the position of an object. The optical position sensor may use the emission and detection of light signals to calculate the position and movement. The optical position sensor may track markers or features on the medical tool 108 and determine its position relative to the physical model 101. A magnetic position sensor, such as a Hall effect sensor or a magnetoresistive sensor, may utilize magnetic fields to detect the position and movement of objects. The magnetic position sensor may detect the position of the medical tool 108 that is incorporated with magnets or magnetic elements.

The physical model 101 may include one or more haptic sensors 107. The haptic sensor 107 may be also a force sensor or a pressure sensor. A haptic sensor 107 transfers a sense of touch to electrical signals during interactions with physical model 101, such as the opening 105 and the canal area 103, by applying forces, vibrations, or motions. The haptic sensor 107 may include a force feedback loop that manipulates the deformation of the physical model 101. The haptic sensor 107 can convert mechanical forces such as strain or pressure into electrical signals. The haptic sensor 107 may rely on a combination of force, vibration, and motion to recreate the sense of touch. The haptic sensor 107 may include, without limitation, a fiber bragg gratings (FBGs) sensor, an eccentric rotating mass vibration (ERMV) sensor, a linear resonant actuator (LRAs) sensor, or a piezo haptic sensor. The haptic sensor 107 may attach to the surface of the canal area 103 or anywhere that may detect a force or a pressure applied to the physical model 101.

The disclosed embodiments may include one or more medical tools 108. A medical tool 108 is any instrument, apparatus, implement, machine, appliance, implant, reagent for in vivo use, software, material or other similar or related article, intended by the manufacturer to be used, alone or in combination for a medical purpose. The medical tool 108 may be, without limitations, a surgical instrument (such as scalpels, scissors, saws, forceps, clamps, cautery, retractors, or lancets), a catheter, a cannula, an endoscope, an injection device, a laparoscopic instrument, or any medical tool that is suitable for practice on the physical model 101 to simulate clinical procedures. In embodiments, the medical tool 108 may be operable to insert through the opening 105 into the canal area 103. In embodiments, the medical tool may be a speculum, a tenaculum, or an IUD inserter. The speculum, the tenaculum, or the IUD inserter may be used in gynecological and obstetric procedures. The speculum may be used to visualize and access the cervix and vaginal canal. The tenaculum may be used to hold and stabilize tissues during gynecological procedures, such as colposcopy or cervical biopsies. The IUD inserter may be used to place an intrauterine device into the uterus. The IUD inserter may include a long, slender tube and a plunger-like mechanism. The IUD may be loaded into the IUD inserter. The slender tube may be inserted through the cervical canal into the uterus and the plunger-like mechanism may be pushed to release the IUD to expand and position itself within the uterus.

The clinical procedure training system 100 may include one or more cameras 208. The camera 208 may be operable to acquire image and video data of the physical model 101, the medical tool 108, and the real-world environment around the physical model 101 and the medical tool 108. The camera 208 may be, without limitation, a RGB camera, a depth camera, an infrared camera, a wide-angle camera, or a stereoscopic camera. The camera 208 may be equipped, without limitations, on a smartphone, a tablet, a computer, a laptop, or a virtual head unit 120. The clinical procedure training system 100 may include one or more display 209. The display 209 may be equipped, without limitations, on a smartphone, a tablet, a computer, a laptop, or a virtual head unit 120, such as augmented reality (AR) glasses.

The clinical procedure training system 100 may include one or more virtual head units 120. The virtual head unit 120 may include a camera 208, a display 209, glasses 122, a tracking sensor, a processor 204, and a projector 124. The virtual head unit 120 may be used for Augmented Reality (AR), Mixed Reality (MR), Extended Reality (XR or X-reality), holography (image overlay), and artificial intelligence (AI) in immersive virtual worlds and combines virtual reality (VR) and augmented reality (AR) technologies to provide an immersive and interactive user experience. In embodiments, the virtual head unit 120 is a see-through display or AR glasses to be worn on the head of a user. The projector 124 may cast virtual images on the glasses or directly onto the user's eyes to be superimposed onto the user's vison such that the virtual images are combined with the real-world view. The tracking sensor, such as, without limitations, an infrared sensor, an accelerometer, a gyroscope, or an external tracking system, may monitor the user's head movements and position. The tracking sensor can use various technologies to track the user's movements.

The clinical procedure training system 100 may include one or more processors. The processor may be included, without limitations, in the controller 201 (such as a computer, a laptop, a tablet, a smartphone, or a medical equipment), the virtual head unit 120, a server, or a third-party electronic device.

In one embodiment, the clinical procedure training system 100 may include a laptop with a simulator software installed, a systems electronics unit, a pelvic model, medical tools, such as an IUD inserter, a speculum, a tenaculum, and/or a sound. The sound may be used to gauge the depth and position of a uterine cavity in the canal area 103 for adjustment of a flange on the IUD inserter for desired deployment. The system's electronics unit may include a plurality of input sockets operable to be connected to the medical tools and the pelvic model.

Referring to FIG. 2, example non-limiting components of the clinical procedure training system are depicted. The clinical procedure training system 100 may include a controller 201. The controller 201 may include various modules. For example, the controller 201 may include a mixed reality module 222, a feedback module 232, and a recommendation module 242. The controller 201 may further comprise various components, such as a memory component 202, a processor 204, an input/output hardware 205, a network interface hardware 206, a data storage component 207, and a local interface 203. The controller 201 may include a camera 208 and a display 209.

The controller 201 may be any device or combination of components comprising a processor 204 and a memory component 202, such as a non-transitory computer readable memory. The processor 204 may be any device capable of executing the machine-readable instruction set stored in the non-transitory computer readable memory. Accordingly, the processor 204 may be an electric controller, an integrated circuit, a microchip, a computer, or any other computing device. The processor 204 may include any processing component(s) configured to receive and execute programming instructions (such as from the data storage component 207 and/or the memory component 202). The instructions may be in the form of a machine-readable instruction set stored in the data storage component 207 and/or the memory component 202. The processor 204 is communicatively coupled to the other components of the controller 201 by the local interface 203. Accordingly, the local interface 203 may communicatively couple any number of processors 204 with one another, and allow the components coupled to the local interface 203 to operate in a distributed computing environment. The local interface 203 may be implemented as a bus or other interface to facilitate communication among the components of the controller 201. In some embodiments, each of the components may operate as a node that may send and/or receive data. While the embodiment depicted in FIG. 2 includes a single processor 204, other embodiments may include more than one processor 204.

The memory component 202 (e.g., a non-transitory computer-readable memory component) may comprise RAM, ROM, flash memories, hard drives, or any non-transitory memory device capable of storing machine-readable instructions such that the machine-readable instructions can be accessed and executed by the processor 204. The machine-readable instruction set may comprise logic or algorithm(s) written in any programming language of any generation (e.g., 1GL, 2GL, 3GL, 4GL, or 5GL) such as, for example, machine language that may be directly executed by the processor 204, or assembly language, object-oriented programming (OOP), scripting languages, microcode, etc., that may be compiled or assembled into machine readable instructions and stored in the memory component 202. Alternatively, the machine-readable instruction set may be written in a hardware description language (HDL), such as logic implemented via either a field-programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components. For example, the memory component 202 may be a machine-readable memory (which may also be referred to as a non-transitory processor-readable memory or medium) that stores instructions that, when executed by the processor 204, causes the processor 204 to perform a method or control scheme as described herein. While the embodiment depicted in FIG. 2 includes a single non-transitory computer-readable memory component, other embodiments may include more than one memory module. The memory may be used to store the mixed reality module 222, the feedback module 232, and the recommendation module 242. Each of the mixed reality module 222, the feedback module 232, and the recommendation module 242 during operating may be in the form of operating systems, application program modules, and other program modules. Such program modules may include, but are not limited to, routines, subroutines, programs, objects, components, and data structures for performing specific tasks or executing specific abstract data types according to the present disclosure as will be described below.

The input/output hardware 205 may include a monitor, keyboard, mouse, printer, camera, microphone, speaker, and/or other device for receiving, sending, and/or presenting data. The network interface hardware 206 may include any wired or wireless networking hardware, such as a modem, LAN port, Wi-Fi card, WiMax card, mobile communications hardware, and/or other hardware for communicating with other networks and/or devices.

The data storage component 207 stores collected visualization data, data generated by the sensors, and data of operating microelectrodes, heaters, and coils. The mixed reality module 222, the feedback module 232, and the recommendation module 242 may also be stored in the data storage component 207 during operating or after operation.

Each of the mixed reality module 222, the feedback module 232, and the recommendation module 242 may include one or more machine learning algorithms or neural networks. The mixed reality module 222, the feedback module 232, and the recommendation module 242 may be trained and provided machine learning capabilities via a neural network as described herein. By way of example, and not as a limitation, the neural network may utilize one or more artificial neural networks (ANNs). In ANNs, connections between nodes may form a directed acyclic graph (DAG). ANNs may include node inputs, one or more hidden activation layers, and node outputs, and may be utilized with activation functions in the one or more hidden activation layers such as a linear function, a step function, logistic (sigmoid) function, a tanh function, a rectified linear unit (ReLu) function, or combinations thereof. ANNs are trained by applying such activation functions to training data sets to determine an optimized solution from adjustable weights and biases applied to nodes within the hidden activation layers to generate one or more outputs as the optimized solution with a minimized error. In machine learning applications, new inputs may be provided (such as the generated one or more outputs) to the ANN model as training data to continue to improve accuracy and minimize error of the ANN model. The one or more ANN models may utilize one-to-one, one-to-many, many-to-one, and/or many-to-many (e.g., sequence to sequence) sequence modeling. The one or more ANN models may employ a combination of artificial intelligence techniques, such as, but not limited to, Deep Learning, Random Forest Classifiers, Feature extraction from audio, images, clustering algorithms, or combinations thereof. In some embodiments, a convolutional neural network (CNN) may be utilized. For example, a convolutional neural network (CNN) may be used as an ANN that, in a field of machine learning, for example, is a class of deep, feed-forward ANNs applied for audio analysis of the recordings. CNNs may be shift or space invariant and utilize shared-weight architecture and translation. Further, each of the various modules may include a generative artificial intelligence algorithms. The generative artificial intelligence algorithm may include a general adversarial network (GAN) that has two networks, a generator model and a discriminator model. The generative artificial intelligence algorithm may also be based on variation autoencoder (VAE) or transformer-based models.

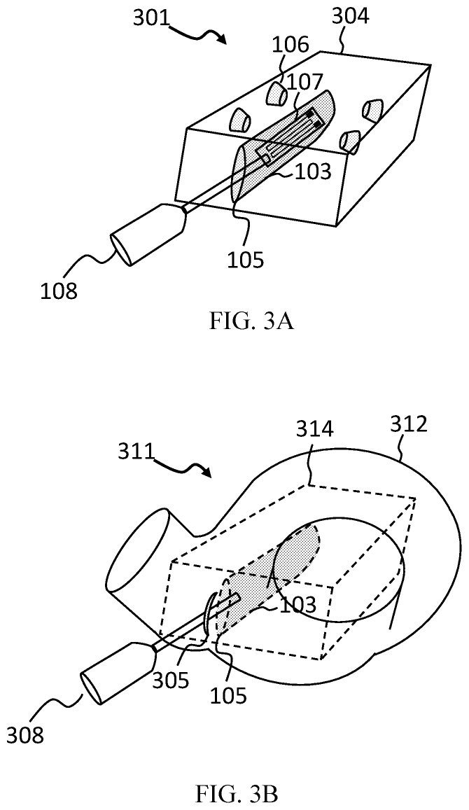

Referring to FIGS. 3A and 3B, the operation of a medical tool on a physical model and simulated virtual representation of the operation of the medical tool on the physical model are depicted. A user who operates the physical model 304 and medical tool 108 may apply the medical tool 108 to the physical model 304 as if operating the medical tool 108 on an anatomic region represented by the physical model 304. The medical tool may be a speculum, a tenaculum, an intrauterine device (IUD) inserter, or any other medical tool appropriate and suitable for operation on vagina, cervix, uterus, ovaries, and fallopian tubes during gynecological and obstetric procedures. The speculum may be used to visualize and access the cervix and vaginal canal. The tenaculum may be used to hold and stabilize tissues during gynecological procedures, such as colposcopy or cervical biopsies. The IUD inserter may be used to place an intrauterine device (IUD) into the uterus. The IUD inserter may include a long, slender tube and a plunger-like mechanism. The IUD may be loaded into the IUD inserter. The slender tube may be inserted through the cervical canal into the uterus and the plunger-like mechanism may be pushed to release the IUD to expand and position itself within the uterus.

In embodiments, the controller 201 may provide instructions and steps on the display 209 to operate the medical tool 108, such as the speculum, the tenaculum, or the IUD inserter on a physical model 304, such as a pelvic model. For example, the controller 201 may provide instruction and steps on the display 209 to instruct the user to practice an appropriate position for an actual patient during an IUD insertion procedure. The controller 201 may instruct the user to orient the physical model 304 to facilitate desired access and visualization of the opening 105 as an insertion site. Further, the controller 201 may instruct the user to visually identify and locate the relevant anatomical landmarks on the physical model 304, such as the cervix or uterine cavity. The controller 201 may instruct the user to follow more steps provided on the display 209 for inserting the IUD inserter into the canal area 103 and placing and releasing the IUD at the desired place within the canal area 103, such as an simulated uterine cavity. During the process, the position sensors 106 and the haptic sensor 107 may monitor the position of the medical tool and the force or pressure applied to the walls of the canal area 103 in real time.

As shown in FIG. 3A, during the operation of the medical tool 108 and the physical model 304, the controller 201 may display a physical view 301 of the operation of the medical tool 108 on the physical model 304, such as a pelvic model, as illustrated. The physical view 301 does not include virtual representation 312 and may help the user to perceive the actual environment during the operation. The physical view 301 may be captured by the camera 208, which may be equipped on the virtual head unit 120 (e.g. as illustrated in FIG. 1) or on other electronic devices, such as a tablet, a smartphone, a laptop, a computer, or the like. The physical view 301 may include elements of the physical model 304 that a user may not see directly through the user's eyes, such as the position sensors 106, the canal area 103, and the haptic sensor 107. Such a physical view may help the user to apprehend the position of the medical tool 108 inside the physical model 304 and further adjust the operation accordingly.

As shown in FIG. 3B, a simulated view 311 of the operation of the medical tool 108 on the physical model 304, such as a pelvic model, is illustrated. The simulated view 311 may include both virtual representation 312 and the physical model 304. The virtual representation 312 may including the anatomic region represented by the physical model 304 and the interactions between the anatomic region with the medical tool 108. The virtual representation 312, such as a real-time virtual representation, may be generated by overlaying an anatomic image on the physical model 314 and the medical tool 308. The simulated view 311 may include the physical model 314 overlaying with the virtual representation 312. In embodiments, the virtual representation 312 may include the simulated opening 305.

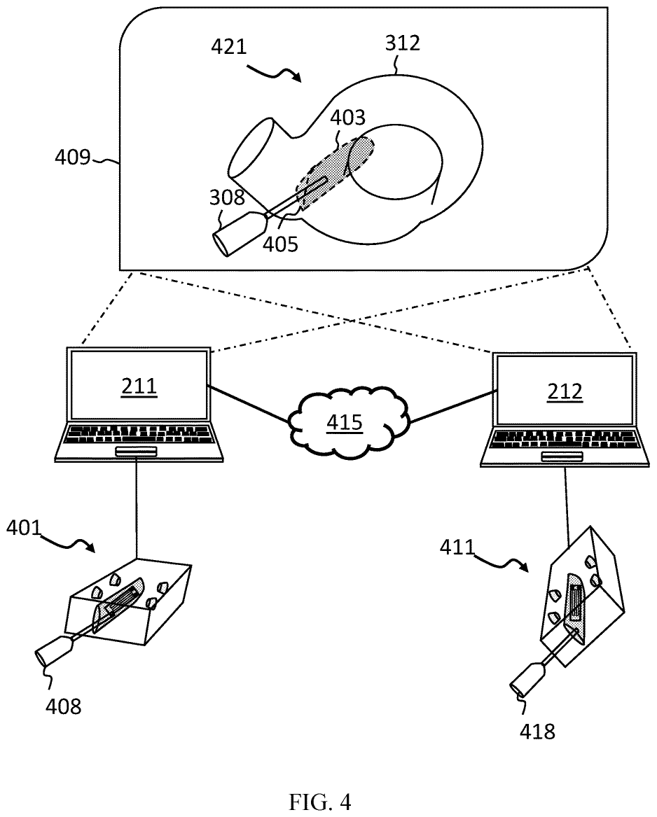

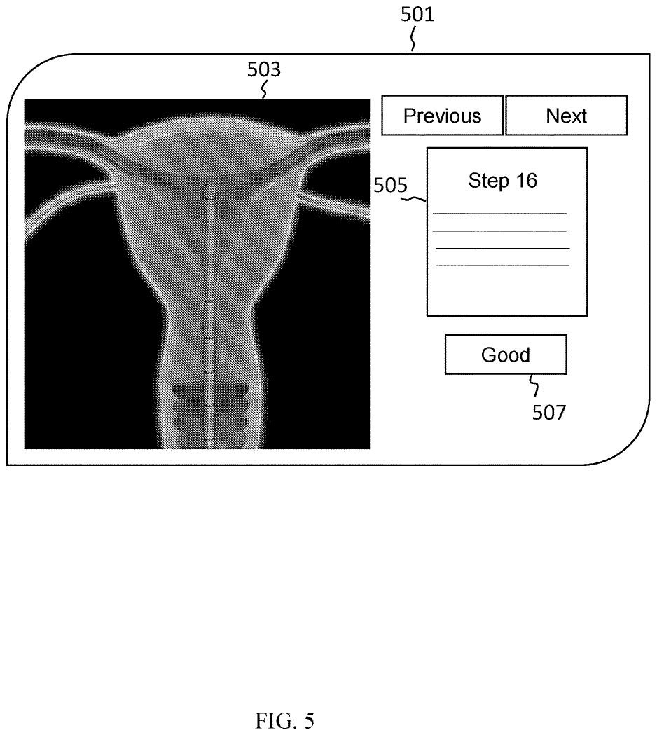

The controller 201 may use the mixed reality module 222 (e.g. as illustrated in the FIG. 2) to generate the virtual representation 312 that is overlaid with the physical model 314 and the medical tool 108 in the real-world environment. The controller 201 may include anatomic images 227 and digital three dimensional (3D) models that are built based on 3D modeling and 3D software, which are stored in the data storage component 207 (e.g. as illustrated in FIG. 2). The controller 201 may track the positions and orientations of the physical model 314 and the medical tool 108 using optical tracking or markers based on data collected by the cameras 208, position sensors 106. The controller 201 may then align and map the positions and orientations of the anatomic images 227 and the digital 3D models with the physical model 314 and the medical tool 108 to generate simulated representations in the simulated view 311, which may include the anatomic regions, the simulated medical tool 308. The controller 201 may further continuously calibrate virtual space to the real-world space and align the tracking data with the physical model 314, creating an augmented or mixed reality experience. The controller 201 may selectively include the real-world components of physical view 301 in the simulated view 311, such as the physical model 314 and the opening 105 as illustrated in FIGS. 3B and 4. The controller 201 may also exclude the real-world components of physical view 301, leaving only virtual representations displayed to the user, for example, as illustrated in FIG. 5.

Referring to FIG. 1 and FIG. 4 together, an exemplary operation of a plurality of medical tools on a physical model for shared mixed reality is depicted. Two or more users may perform operations or practices over two or more physical models 101 and/or two or more medical tools 108 (e.g. as illustrated in FIG. 1), for example, a first physical model 401, a second physical model 411, a first medical tool 408, and a second medical tool 418. The operations or practices may be performed locally or in distance. Sensor data may be generated in real time by sensors such as the position sensors 106 and haptic sensors 107 of the first physical model 401, the second physical model 411, the first medical tool 408, and the second medical tool 418. Images and videos of operation of each physical model 401, 411 and each medical tool 408, 418 may be captured by one or more cameras 208 (e.g. as illustrated in FIGS. 1 and 2). The clinical procedure training system 100 may include two or more controllers 201 as peer controllers, such as a first controller 211 and a second controller 212. The peer controllers may be connected to each other through a connection 415. The peer controllers may be further connected to a server or a third-party controller via the connection 415. A connection 415 may be wired or wireless, such as Ethernet, local area network, universal serial bus (USB), WiFi, Bluetooth, near field communication, infrared short-range wireless communication, internet, or mobile network (e.g. 2G, 3G, 4G, 5G, and 6G mobile network).

The sensor data, images and videos of operation of the physical models 101 and medical tools 108 may be collected and transmitted to the first controller 211 and the second controller 212. The controllers 201 in the clinical procedure training system 100, including the first controller 211 and the second controller 212, serve as peer controllers. Each controller 201 has the capability to establish local connections and independently control the electronic components within the system, such as sensors and cameras. A peer controller may transmit the sensor data, images and videos via a connection 415 to another peer controller. A peer controller may also transmit the sensor data, images and videos to a server or a third-party controller. In embodiments, after a peer controller receives the sensor data, images, and videos of the operation, the peer controller, such as the first controller 211 and the second controller 212, may generate the shared mixed reality image or video 421 asynchronously or synchronously. The generated shared mixed reality image or video 421 may be displayed at a local display 409, such as a display 209 or virtual head unit 120. The local display 409 is associated with the peer controller generating the shared mixed reality image or video. In some embodiments, a server or a third-party controller may generate the shared mixed reality image or video 421 asynchronously or synchronously and further transmit the generated shared mixed reality image or video 421 via the connection 415 to the peer controllers for display at the local display 409.

The clinical procedure training system 100 may include a data fusion algorithm to integrate multiple data sources available in the clinical procedure training system 100 to produce consistent and desired information, such as the shared mixed reality image or video 421. After the sensor data, the images and/or the videos are collected locally and received from a peer controller, a peer controller or a serve may use the data fusion algorithm to fuse the collected data, such as the sensor data, the image, and/or the videos to generate the shared mixed reality image or video. The data fusion algorithm may align the sensor data, images and videos in terms of their timing and spatial information of the one or more physical models and the one or more medical tools, such as the first physical model 401, the second physical model 411, the first medical tool 408, and the second medical tool 418. The data fusion algorithm may then fuse the collected data using, without limitations, a complementary fusion, a redundant fusion, a cooperative fusion, a competitive fusion. The data fusion algorithm may use different approach to fuse the collected data, such as, without limitations, Kalman filtering, Bayesian inference, sensor weighting, feature-based fusion, or data association.

In some embodiments, the first medical tool 408 and the second medical tool 418 may be a same medical tool, such as a speculum, a tenaculum, or an IUD inserter. In such scenario, the users using the same medical tool may compete with each other according to the instruction provided by the controller 201. The users may provide example operation showing to another user how to operate the medical tool. One or more shared mixed reality images or videos 421 may be displayed on the local display 409 presenting the operation of medical tool 308 of different users. For example, as illustrated in FIG. 4, a simulated virtual representation, such as the shared mixed reality image or video 421, represents a medical tool 308 such as a IUD inserter, inserted into the simulated canal area 403 through the simulated opening 405. A user may switch between the shared mixed reality images or videos 421 or display them side by side on the local display 409 to compare their differences.

In some embodiments, the first medical tool 408, and the second medical tool 418 may be different medical tools. In such a scenario, the users may use different medical tools to cooperate with each other to perform a medical procedure requiring two or more medical tools. A shared mixed reality image or video 421 may be displayed on the local display 409 presenting the operation of two and more medical tools 308, such as the first medical tool 408 and the second medical tool 418 of different users. For example, a simulated virtual representation, such as a shared mixed reality image or video 421, may include the first medical tool 408 such as a speculum, which is operated by a first user to keep the simulated opening 405 in a desired shape for a IUD inserted to be inserted into the simulated canal area 403 through the simulated opening 405, and the second medical tool 418, such as an IUD inserter, inserted into the simulated canal area 403 through the simulated opening 405 at a desired shape. The shared mixed reality image or video 421 may display the interactions between the simulated physical model as in the virtual representation 312 and the medical tools 308, and also between the first medical tool 408 and the second medical tool 418.

Referring to FIG. 5, an exemplary user interface of the clinical procedure training system is depicted. The user interface 501 may be displayed on the display 209 of a controller 201, a table, a smartphone, a laptop, a computer, or a virtual head unit 120 (e.g. as illustrated in FIG. 1), or projected onto glasses or users' eyes by the projector of the virtual head unit 120 (e.g. as illustrated in FIG. 1). The user interface 501 may include a region displaying a virtual representation 503 generated based on AR, MR, XR, or holography, or images and videos captured by the cameras, or any stored images or videos on the controller 201. The user interface 501 may include an region displaying information of the clinical procedure for the training, such as step information (e.g., “step 16”), an instruction of operation in the steps, an image corresponding to the current step. The user interface may further include a region displaying a feedback 507 of the operation in the current step, in a historical step, in an overall operation, in an historical operation, of the current user or any previous users. The user interface 501 may further include a control interaction region for control the software, allowing the user to advance to the next step, revert to a previous step, or load a menu which will allow the application to be restarted or exited. The user interface 501 may further include an alert region that may change colors and may display text indicating potential problems with the user's performance in some or all steps.

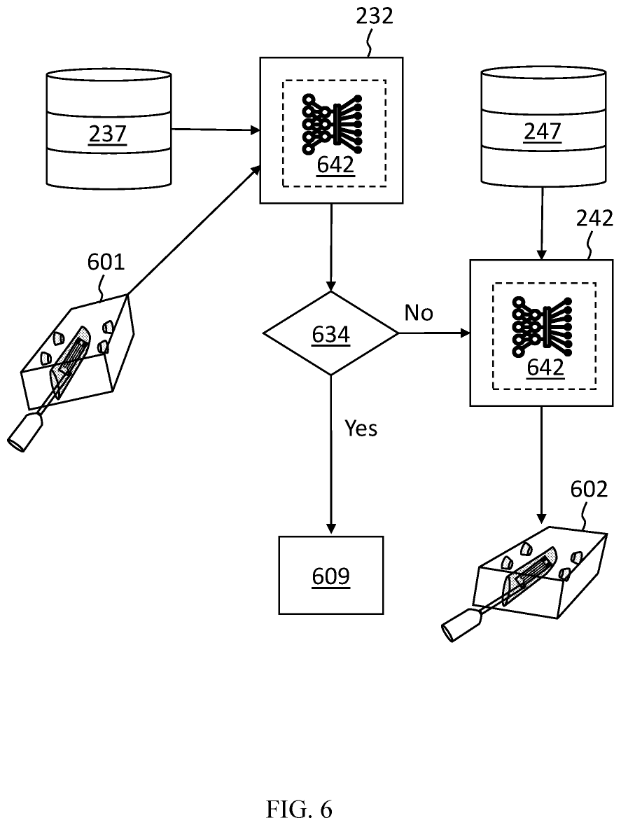

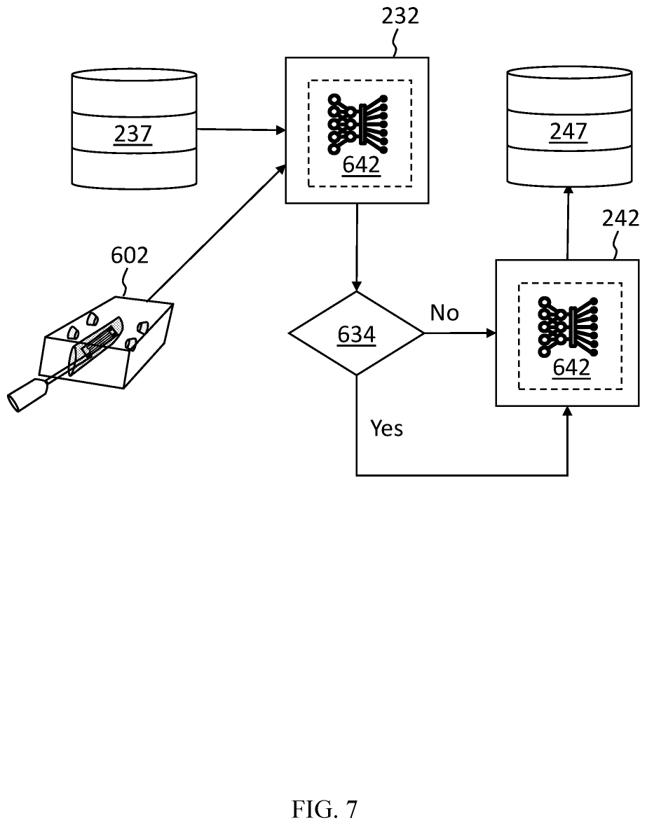

Referring to FIGS. 6 and 7, block diagrams of the method for clinical procedure training and the method for training the clinical procedure training system are depicted. The current operation 601 of the medical tool by a user is analyzed by the feedback module 232 of the controller 201 (e.g. as illustrated in FIG. 2) to determine if the current operation 601 satisfies the requirements provided in the clinical procedure training system 100. In embodiments, the feedback module 232 may include one or more tolerance thresholds and apply a learning model 642 in comparing the operation of the user and the desired operation and determining whether the operation of the user satisfies the requirements. For example, the feedback module 232 may apply the learning model 642 to compare the location of the medical tool with a procedure location associated with a current step and an applied pressure by the medical tool on the canal area and a procedure applied pressure. Accordingly, the feedback module 232 may provide feedback 507 to the user on the user interface 501 (e.g. as illustrated in FIG. 5), such as “Good.” In some embodiments, the feedback may include location and trajectory feedback and tissue pressure feedback. The location and trajectory feedback may reflect the difference between the current location of the medical tool as detected by the position data and a procedure location provided in the feedback module 232. The tissue pressure feedback may reflect the difference between the current applied pressure to the canal area detected by the haptic sensor and a procedure applied pressure provided in the feedback module 232. For example, a location and trajectory feedback may be “The depth of insertion is exceeded by 1 cm.” A tissue pressure feedback may be “It is important to apply gentle and steady force while inserting the IUD inserter, as exceeding a patient's tolerance can be uncomfortable.” In embodiments, the feedback module 232 may determine the feedback by further comparing the current operation 601 with the historical training performance of the user stored in the historical training performance data 237. A feedback may be displayed in the user interface 501 that reflects the historical performance of the user, such as “Congratulations on your improvement! Your operation now meets the standard of “Good”.”

In embodiments, the learning model 642 may include a machine-learning algorithm to determine the performance of an operation. The learning model 642 may be trained based on a dataset containing a wide range of operation data by users, such as positions of the medical tools and applied pressure at various steps and stages. The dataset may include the historical training performance data 237 including the sensor data associated with the performance by the users. The performance may be classified as excellent, good, fair, or fail. The learning model 642 is trained to identify the performance associated with the position of the medical tool, applied pressure to the physical model, and other sensor data, considering the historical operation by a wide range of users. The training effectiveness of the machine learning algorithm is validated using multiple evaluation metrics, such as precision, recall, and accuracy. The training process can be evaluated by the system using predetermined threshold metrics until the desired level of accuracy is achieved through training. The desired level of accuracy may be denoted as confidence level, a value between 0 and 1. The trained learning model 642 may be continuously validated with the current operation 601 in association with feedback from the users.

The feedback module 232 may determine 634 whether the performance satisfies the requirements provided by the feedback module 232. If the answer is yes (yes to satisfying requirements 334), a positive feedback may be displayed on the user interface 501, such as “Good.” Conversely, if the answer is no (no to satisfying requirements 334), the performance associated with the current operation 601, the sensor data, the images and/or videos of the physical model and medical tool may be fed to the recommendation module 242 to generate personalized feedback and recommendation, such as additional training 602, to be displayed on the user interface 501. The recommendation module 242 may make the recommendation, such as additional training 602, based on the performance and current operation 601 of the user and the historical recommendation data 247. The historical recommendation data 247 may include data about the historical performance of users and whether there was any improvement after implementing the recommendations.

In embodiments, the recommendation module 242 may recommend additional training 602 using a learning model 642. The learning model 642 may incorporate games and offers different modes and configurations for training. For example, the learning model 642 may include synchronous one-to-one with instructor model, a synchronous one-to-many learners model, an asynchronous one instructor, multiple groups model, a local co-location of instructor and learners model, and a distance separated remote model. In the synchronous one-to-one with instructor model, a single user may interact directly with an instructor in real-time. The instructor may provide personalized guidance, feedback, and instruction tailored to the user's needs. In the one-to-many learners model, multiple users may participate in the training session simultaneously, remotely or locally, interacting with the instructor and each other. The instructor delivers instructions, facilitates discussions, and coordinates activities for the entire group. In the one-instructor-multiple-groups model, an instructor may guide multiple groups of users who are learning the same skill but at different times (asynchronously). Each group may progress through the training program independently, the instructor provides resources, assignments, and assessments tailored to each group's pace and progress. In the co-location-of-instructor-and-learners model, both the instructor and users may be physically present in the same location, such as a classroom or training facility. In the distance-separated model, the instructor and users may be geographically separated, engaging in training remotely. Communication and interaction occur through the clinical procedure training system 100.

The recommendation module 242 may recommend additional training 602, which may include gaming, competition, and cooperation. For the gaming, the recommendation module 242 may introduce game elements, such as scoring, levels, achievements, or challenges to the additional training 602. For the competition, the recommendation module 242 may introduce competitive elements to drive the user to enhance her skills. For example, the recommendation module 242 may introduce leaderboards, timed challenges, or performance-based assessment in the additional training 602. For the cooperation, the recommendation module 242 may ask another user to cooperate with the user being recommended for additional training 602 to perform group-based activities, such as team challenges. The recommendation module 242 may provide multiple options for additional training 602 and allow the user to select from the options. For example, the recommendation module 242 may provide options to the user by challenging the user to (1) “Redo step 16 in 2 minutes without an improper twist” (an observed mistake made during the last operation), (2) “Redo step 16 with the cooperating user A and share your experience after redoing.” The user may then take an option of the recommendation, such as additional training 602. The recommendation module 242 may allow the user to move forward to a further step without additional training 602. In some embodiments, the recommendation module 242 may block the further step unless additional training 602 is done.

Referring to FIG. 7, the method for continuously training the clinical procedure training system is depicted. The recommendation module 242 may include the learning model 642 to make recommendation, such as additional training 602. The learning model 642 may be trained on a dataset containing a wide range of user performances along with recommendations given by the recommendation module 242. For example, once a user accepts additional training 602 and performs the operation, the clinical procedure training system 100 may collect the sensor data, images and videos during the additional training 602 to be fed to the feedback module 232. The feedback module 232 may compare with the historical training user performance data 237 and determine 634 whether the performance of the user during the additional training 602 satisfies the requirements provided by the feedback module 232, and whether performance improvements are made. A positive (Yes to 634) or a negative (No to 634) evaluation, along with the additional training 602 may be fed to the recommendation module 242 for training and validation. Further, the trained recommendation module 242 may be continuously validated with the additional training 602 association with feedback from the user.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including matters of logic with respect to the arrangement of steps, operational flow, order of components, or orientation of components; plain meaning derived from grammatical organization or punctuation, and; the number or type of embodiments described in the specification.

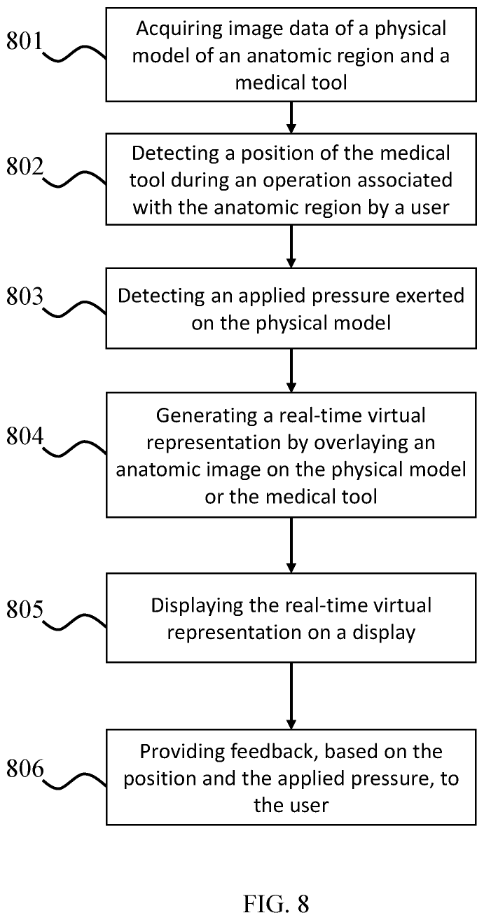

FIG. 8 illustrates a flow diagram of illustrative steps for clinical procedure training. At block 801, the method for clinical procedure training may include acquiring, using a camera, image data of a physical model of an anatomic region and a medical tool operable to interact with the physical model. In embodiments, the medical model may be, without limitation, a skeletal anatomy model, a muscular anatomy model, an organ anatomy model, a skull anatomy model, a torso anatomy model, a joint model, a vascular model, or a full-body anatomical model. The physical model may be a pelvic model including a canal area having an opening at a surface of the pelvic model. The medical tool is operable to insert through the opening into the canal area. In embodiments, the medical tool may be a bone saw used to demonstrate and practice bone dissection techniques, a bone forceps used for gripping and manipulating bones, a bone drill used to simulate drilling holes for orthopedic procedures, a muscle biopsy needle (such as a traditional Bergstrom needle) used to practice muscle biopsy procedures, forceps used for practicing to grasp, retract, or stabilize tissue, dental tools such as dental probes or dental extraction forceps for practicing dental procedures, otoscope for practicing examination of the ear canal and eardrum, or ophthalmoscope used for examining the interior of the eye. The medical tool may be, without limitations, a speculum, a tenaculum, or an intrauterine device (IUD) inserter. At block 802, the method for clinical procedure training may include detecting, using a position sensor, a position of the medical tool during an operation associated with the anatomic region by a user. At block 803, the method for clinical procedure training may include detecting, using a haptic sensor, an applied pressure exerted on the physical model, such as on the canal area 103 (as illustrated in FIG. 1). At block 804, the method for clinical procedure training may include generating a real-time virtual representation by overlaying an anatomic image on the physical model or the medical tool, such as a simulated view 311 (as illustrated in FIGS. 3B, 4, and 5). At block 805, the method for clinical procedure training may include displaying the real-time virtual representation on a display. The display 209 may be equipped on a controller 201 or a virtual head unit 120 (e.g., as illustrated in FIG. 1).

At block 806, the method for clinical procedure training may include providing feedback, based on the position and the applied pressure, to the user. The feedback may include location and trajectory feedback and tissue pressure feedback. The location and trajectory feedback may be determined based on a comparison between the location and a procedure location. The tissue pressure feedback may be determined based on a comparison between the applied pressure and a procedure applied pressure.

In embodiments, the method for clinical procedure training may further include tracking training performance of the user based on operation time and a difference between the position and a procedure position of the medical tool, applying the learning model to the training performance to determine whether to recommend additional training, after determining to recommend the additional training, providing personalized training to the user based on the learning model, wherein the personalized training include gaming, competition, and cooperation.

In embodiments, the method for clinical procedure training may further include training the learning model using the training performance of the user during the personalized training form and instrument.

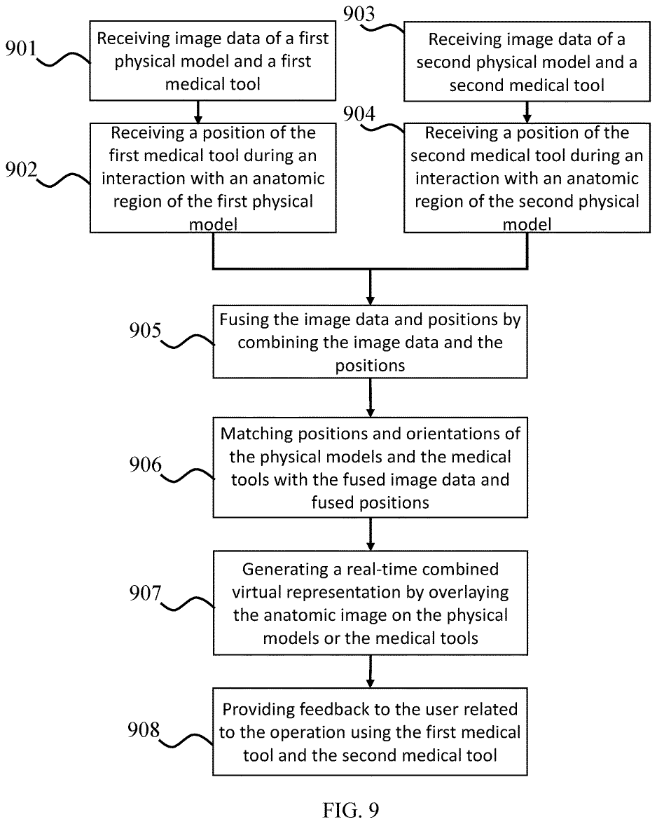

FIG. 9 illustrates a flow diagram of illustrative steps for multiparty clinical procedure training. At block 901, the method for multiparty clinical procedure training may include receiving image data of a first physical model and a first medical tool. The first medical tool may include, but is not limited to, a speculum, a tenaculum, or an IUD inserter. At block 902, the method for multiparty clinical procedure training may include receiving a position of the first medical tool during an interaction with an anatomic region of the first physical model. At block 903, the method for multiparty clinical procedure training may include receiving image data of a second physical model and a second medical tool. The second medical tool may include, but is not limited to, a speculum, a tenaculum, or an IUD inserter. At block 904, the method for multiparty clinical procedure training may include receiving a position of the second medical tool during an interaction with an anatomic region of the second physical model. At block 905, the method for multiparty clinical procedure training may include fusing the image data and positions by combining the image data of the first physical model, the first medical tool, the second physical model, the second medical tool, and the positions of the first medical tool and the second medical tool. At block 906, the method for multiparty clinical procedure training may include matching positions and orientations of the first physical model, the second physical model, the first medical tool, and the second medical tool with the fused image data and fused positions. At block 907, the method for multiparty clinical procedure training may include generating a real-time combined virtual representation by overlaying the anatomic image on the first physical model, the first medical tool, the second physical model, or the second medical tool. At block 907, the method for multiparty clinical procedure training may include displaying the real-time combined virtual representation on the display. The real-time combine virtual representation may be a shared mixed reality image or video 421 (e.g. as illustrated in FIG. 4). At block 908, the method for multiparty clinical procedure training may include providing feedback to the user related to the operation using the first medical tool and the second medical tool.

In embodiments, the method for multiparty clinical procedure training may further include receiving an applied pressure exerted on the anatomic region of the second medical tool and determining forces and frictions between the first medical tool and the second medical tool based on the positions of the first medical tool and the second medical tool, and the applied pressures associated with the first medical tool and the second medical tool. The forces and frictions between the first medical tool and the second medical tool may be determined based on the interactions, the contact surfaces between the medical tools, the friction coefficients of the first medical tool and the second medical tool, and position and the orientation of the medical tools in the canal area. The feedback provided to the user may include tissue pressure feedback and medical tool force feedback. The medical tool force feedback may include whether the forces and friction between the first medical tool and the second medical tool surpasses a threshold value of the uncomfortableness of an average patient, which may be determined based on a dataset including a wide range of operations of the medical tools applying on patients.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

Further aspects of the embodiments described herein are provided by the subject matter of the following numbered clauses:
1. A system for clinical procedure training comprising a physical model of an anatomic region, wherein the physical model comprises a position sensor and a haptic sensor, a medical tool operable to interact with the physical model, a camera, a display, a processor, and a computer-readable medium storing computer-readable instructions that cause the processor to acquire, using the camera, image data of the physical model and the medical tool, detect, using the position sensor, a position of the medical tool during an operation associated with the anatomic region by a user, detect, using the haptic sensor, an applied pressure exerted on the physical model, generate a real-time virtual representation by overlaying an anatomic image on the physical model or the medical tool, display the real-time virtual representation on the display, and provide feedback to the user based on the position and the applied pressure.
2. The system according to clause 1, wherein the physical model comprises a canal area having an opening at a surface of the physical model, and the medical tool is operable to insert through the opening into the canal area.
3. The system according to any previous clause, wherein the physical model is a skeletal anatomy model, a muscular anatomy model, an organ anatomy model, a skull anatomy model, a torso anatomy model, a joint model, a vascular model, or a full-body anatomical model.
4. The system according to any previous clause, wherein the feedback comprises location and trajectory feedback based on a comparison between the location and a procedure location, and tissue pressure feedback based on a comparison between the applied pressure and a procedure applied pressure.
5. The system according to any previous clause, wherein the medical tool is a speculum, a tenaculum, an intrauterine device (IUD) inserter, a surgical instrument, a catheter, a cannula, an endoscope, an injection device, a laparoscopic instrument, a drill, dental tools, an otoscope, or an ophthalmoscope.
6. The system according to any previous clause, wherein the computer-readable instructions further cause the processor to receive image data of a second physical model and a second medical tool, receive a position of the second medical tool during an interaction with an anatomic region of the second physical model, fuse the image data and positions by combining the image data of the physical model, the medical tool, the second physical model, the second medical tool, and the positions of the medical tool and the second medical tool, match positions and orientations of the physical model, the second physical model, the medical tool, and the second medical tool with the fused image data and fused positions, generate a real-time combined virtual representation by overlaying the anatomic image on the physical model, the second physical model, the medical tool, or the second medical tool, display the real-time combined virtual representation on the display, and provide the feedback to the user related to the operation using the medical tool and the second medical tool.
7. The system according to any previous clause, wherein the second medical tool is a speculum, a tenaculum, an IUD inserter, a surgical instrument, a catheter, a cannula, an endoscope, an injection device, a laparoscopic instrument, a drill, dental tools, an otoscope, or an ophthalmoscope.
8. The system according to any previous clause, wherein the computer-readable instructions further cause the processor to receive an applied pressure exerted on the anatomic region of the second medical tool, determine forces and frictions between the medical tool and the second medical tool based on the positions of the medical tool and the second medical tool, and the applied pressures associated with the medical tool and the second medical tool, and wherein the feedback comprises tissue pressure feedback and medical tool force feedback.
9. The system according to any previous clause, wherein the system further comprises a learning model, and the computer-readable instructions further causes the processor to track training performance of the user based on operation time and a difference between the position and a procedure position of the medical tool, apply the learning model to the training performance to determine whether to recommend additional training, and after determining to recommend the additional training, provide a personalized training to the user based on the learning model.
10. The system according to any previous clause, wherein the personalized training comprises gaming, competition, and cooperation.
11. The system according to any previous clause, wherein the computer-readable instructions further cause the processor to train the learning model using the training performance of the user during the personalized training.
12. A method for clinical procedure training comprising acquiring, using a camera, image data of a physical model of an anatomic region and a medical tool operable to interact with the physical model, detecting, using a position sensor, a position of the medical tool during an operation associated with the anatomic region by a user, detecting, using a haptic sensor, an applied pressure exerted on the physical model, generating a real-time virtual representation by overlaying an anatomic image on the physical model or the medical tool, displaying the real-time virtual representation on a display, and providing feedback, based on the position and the applied pressure, to the user.
13. The method according to clause 12, wherein the physical model comprises a canal area having an opening at a surface of the physical model, and the medical tool is operable to insert through the opening into the canal area.
14. The method according to clause 12 and clause 13, wherein the physical model is a skeletal anatomy model, a muscular anatomy model, an organ anatomy model, a skull anatomy model, a torso anatomy model, a joint model, a vascular model, or a full-body anatomical model.
15. The method according to any of clauses 12-14, wherein the feedback comprises location and trajectory feedback based on a comparison between the location and a procedure location, and tissue pressure feedback based on a comparison between the applied pressure and a procedure applied pressure.
16. The method according to any of clauses 12-15, wherein the medical tool is a speculum, a tenaculum, an IUD inserter, a surgical instrument, a catheter, a cannula, an endoscope, an injection device, a laparoscopic instrument, a drill, dental tools, an otoscope, or an ophthalmoscope.
17. The method according to any of clauses 12-16, wherein the method further comprises receiving image data of a second physical model and a second medical tool, receiving a position of the second medical tool during an interaction with an anatomic region of the second physical model, fusing the image data and positions by combining the image data of the physical model, the medical tool, the second physical model, the second medical tool, and the positions of the medical tool and the second medical tool, matching positions and orientations of the physical model, the second physical model, the medical tool, and the second medical tool with the fused image data and fused positions, generating a real-time combined virtual representation by overlaying the anatomic image on the physical model, the second physical model, the medical tool, or the second medical tool, displaying the real-time combined virtual representation on the display, providing the feedback to the user related to the operation using the medical tool and the second medical tool, and wherein the second medical tool is a speculum, a tenaculum, an IUD inserter, a surgical instrument, a catheter, a cannula, an endoscope, an injection device, a laparoscopic instrument, a drill, dental tools, an otoscope, or an ophthalmoscope.
18. The method according to any of clause 17, wherein method further comprises receiving an applied pressure exerted on the anatomic region of the second medical tool, determining forces and frictions between the medical tool and the second medical tool based on the positions of the medical tool and the second medical tool, and the applied pressures associated with the medical tool and the second medical tool, and wherein the feedback comprises tissue pressure feedback and medical tool force feedback.
19. The method according to any of clauses 12-18, wherein the method further comprises tracking training performance of the user based on operation time and a difference between the position and a procedure position of the medical tool, applying a learning model to the training performance to determine whether to recommend additional training, after determining to recommend the additional training, providing a personalized training to the user based on the learning model, and wherein the personalized training comprises gaming, competition, and cooperation.
20. The method according to any of clause 19, wherein the method further comprises training the learning model using the training performance of the user during the personalized training.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

1. A system for clinical procedure training comprising:
a physical model of an anatomic region, wherein the physical model comprises a position sensor and a haptic sensor;
a medical tool operable to interact with the physical model;
a camera;
a display;
a processor; and
a computer-readable medium storing computer-readable instructions that cause the processor to:
acquire, using the camera, image data of the physical model and the medical tool;
detect, using the position sensor, a position of the medical tool during an operation associated with the anatomic region by a user;
detect, using the haptic sensor, an applied pressure exerted on the physical model;
generate a real-time virtual representation by overlaying an anatomic image on the physical model or the medical tool;
display the real-time virtual representation on the display; and
provide feedback to the user based on the position and the applied pressure.

2. The system of claim 1, wherein the physical model comprises a canal area having an opening at a surface of the physical model, and the medical tool is operable to insert through the opening into the canal area.

3. The system of claim 1, wherein the physical model is a skeletal anatomy model, a muscular anatomy model, an organ anatomy model, a skull anatomy model, a torso anatomy model, a joint model, a vascular model, or a full-body anatomical model.

4. The system of claim 1, wherein the feedback comprises:
location and trajectory feedback based on a comparison between the location and a procedure location; and
tissue pressure feedback based on a comparison between the applied pressure and a procedure applied pressure.

5. The system of claim 1, wherein the medical tool is a speculum, a tenaculum, an intrauterine device (IUD) inserter, a surgical instrument, a catheter, a cannula, an endoscope, an injection device, a laparoscopic instrument, a drill, dental tools, an otoscope, or an ophthalmoscope.

6. The system of claim 1, wherein the computer-readable instructions further cause the processor to:
receive image data of a second physical model and a second medical tool;
receive a position of the second medical tool during an interaction with an anatomic region of the second physical model;
fuse the image data and positions by combining the image data of the physical model, the medical tool, the second physical model, the second medical tool, and the positions of the medical tool and the second medical tool;
match positions and orientations of the physical model, the second physical model, the medical tool, and the second medical tool with the fused image data and fused positions;
generate a real-time combined virtual representation by overlaying the anatomic image on the physical model, the second physical model, the medical tool, or the second medical tool;
display the real-time combined virtual representation on the display; and
provide the feedback to the user related to the operation using the medical tool and the second medical tool.

7. The system of claim 6, wherein the second medical tool is a speculum, a tenaculum, an IUD inserter, a surgical instrument, a catheter, a cannula, an endoscope, an injection device, a laparoscopic instrument, a drill, dental tools, an otoscope, or an ophthalmoscope.

8. The system of claim 6, wherein the computer-readable instructions further cause the processor to:
receive an applied pressure exerted on the anatomic region of the second medical tool;
determine forces and frictions between the medical tool and the second medical tool based on the positions of the medical tool and the second medical tool, and the applied pressures associated with the medical tool and the second medical tool; and
wherein the feedback comprises tissue pressure feedback and medical tool force feedback.

9. The system of claim 1, wherein the system further comprises a learning model, and the computer-readable instructions further causes the processor to:
track training performance of the user based on operation time and a difference between the position and a procedure position of the medical tool;
apply the learning model to the training performance to determine whether to recommend additional training; and
after determining to recommend the additional training, provide a personalized training to the user based on the learning model.

10. The system of claim 9, wherein the personalized training comprises gaming, competition, and cooperation.

11. The system of claim 9, wherein the computer-readable instructions further cause the processor to train the learning model using the training performance of the user during the personalized training.

12. A method for clinical procedure training comprising:
acquiring, using a camera, image data of a physical model of an anatomic region and a medical tool operable to interact with the physical model;
detecting, using a position sensor, a position of the medical tool during an operation associated with the anatomic region by a user;
detecting, using a haptic sensor, an applied pressure exerted on the physical model;
generating a real-time virtual representation by overlaying an anatomic image on the physical model or the medical tool;
displaying the real-time virtual representation on a display; and
providing feedback, based on the position and the applied pressure, to the user.

13. The method of claim 12, wherein the physical model comprises a canal area having an opening at a surface of the physical model, and the medical tool is operable to insert through the opening into the canal area.

14. The method of claim 12, wherein the physical model is a skeletal anatomy model, a muscular anatomy model, an organ anatomy model, a skull anatomy model, a torso anatomy model, a joint model, a vascular model, or a full-body anatomical model.

15. The method of claim 12, wherein the feedback comprises:
location and trajectory feedback based on a comparison between the location and a procedure location; and
tissue pressure feedback based on a comparison between the applied pressure and a procedure applied pressure.

16. The method of claim 12, wherein the medical tool is a speculum, a tenaculum, an IUD inserter, a surgical instrument, a catheter, a cannula, an endoscope, an injection device, a laparoscopic instrument, a drill, dental tools, an otoscope, or an ophthalmoscope.

17. The method of claim 12, wherein the method further comprises:
receiving image data of a second physical model and a second medical tool;
receiving a position of the second medical tool during an interaction with an anatomic region of the second physical model;
fusing the image data and positions by combining the image data of the physical model, the medical tool, the second physical model, the second medical tool, and the positions of the medical tool and the second medical tool;
matching positions and orientations of the physical model, the second physical model, the medical tool, and the second medical tool with the fused image data and fused positions;
generating a real-time combined virtual representation by overlaying the anatomic image on the physical model, the second physical model, the medical tool, or the second medical tool;
displaying the real-time combined virtual representation on the display;
providing the feedback to the user related to the operation using the medical tool and the second medical tool; and
wherein the second medical tool is a speculum, a tenaculum, an IUD inserter, a surgical instrument, a catheter, a cannula, an endoscope, an injection device, a laparoscopic instrument, a drill, dental tools, an otoscope, or an ophthalmoscope.

18. The method of claim 17, wherein method further comprises:
receiving an applied pressure exerted on the anatomic region of the second medical tool;
determining forces and frictions between the medical tool and the second medical tool based on the positions of the medical tool and the second medical tool, and the applied pressures associated with the medical tool and the second medical tool; and
wherein the feedback comprises tissue pressure feedback and medical tool force feedback.

19. The method of claim 12, wherein the method further comprises:
tracking training performance of the user based on operation time and a difference between the position and a procedure position of the medical tool;
applying a learning model to the training performance to determine whether to recommend additional training;
after determining to recommend the additional training, providing a personalized training to the user based on the learning model; and
wherein the personalized training comprises gaming, competition, and cooperation.

20. The method of claim 19, wherein the method further comprises training the learning model using the training performance of the user during the personalized training.
